(19) United States
(12) Patent Application Publication
(10) Pub. No.: US 2022/0346691 A1
(43) Pub. Date: Nov. 3, 2022

(54) MUSCLE POTENTIAL MEASUREMENT DEVICE AND MUSCLE POTENTIAL MEASUREMENT CARD

(71) Applicant: SHINKO ELECTRIC INDUSTRIES CO., LTD., Nagano-shi (JP)

(72) Inventor: Hisashi Kaneda, Nagano (JP)

(21) Appl. No.: 17/723,992

(22) Filed: Apr. 19, 2022

(30) Foreign Application Priority Data
Apr. 28, 2021 (JP) 2021-076722

(51) Int. Cl. A61B 5/296; A61B 5/259; A61B 5/389

(52) U.S. Cl. CPC A61B 5/296; A61B 5/259; A61B 5/389; A61B 2562/0209; A61B 2562/227

(57) ABSTRACT

A muscle potential measurement device includes a measurement card including a plurality of interconnects on which pads are formed; a plurality of electrodes that are connected respectively to the pads of part of the interconnects; and a processing circuit that is detachably connected to the measurement card and that is configured to measure a potential that occurs in a subject of measurement with which one ends of the electrodes make contact.

### CROSS-REFERENCE TO RELATED APPLICATION

This application is based upon and claims the benefit of priority of the prior Japanese Patent Application No. 2021-076722, filed on Apr. 28, 2021, the entire contents of which are incorporated herein by reference.

### FIELD

The embodiment discussed herein is related to a muscle potential measurement device and a muscle potential measurement card.

### BACKGROUND

In a conventional medical setting of rehabilitation, or the like, a device that measures a muscle potential that occurs in association with muscle movement is sometimes used. Such a device measures an activity potential that occurs because of activity of muscle fibers by making two or three electrodes make contact with the skin near a muscle that is a subject of measurement.

Electrodes of a muscle potential measurement device include dry electrodes that are fixed to a surface of the device and wet electrodes that are connected to the device via cables. These electrodes make contact with the skin while being separate from each other with appropriate intervals, which makes it possible to measure a muscle potential with high sensitivity.

As a device using dry electrodes, a device in which a plurality of electrodes are arranged on a wearable band that as wearable on a human body and that measures a muscle potential in a way that the wearable band is worn around a muscle that is a subject of measurement is considered.

Patent Literature 1: Japanese Laid-open Patent Publication No. 2016-131689

Patent Literature 2: Japanese Laid-open Patent Publication No. 2018-114093

Patent Literature 3: Japanese Laid-open Patent Publication No. 2015-221144

The above-described muscle potential measurement device however has a problem in that it is difficult to measure a muscle potential of a muscle that is a subject of measurement with sufficient sensitivity. Specifically, the interval between electrodes enabling preferable sensitivity in measurement differs depending on the type of muscle that is a subject of measurement and differs individually. For this reason, in the muscle potential measurement device using the dry electrodes that are arranged fixedly, the interval between electrodes are not necessarily set optimally and there is a limit of increase in sensitivity in measurement. For example, in the muscle potential measurement device using the wet electrodes each of which is independent, while it is possible to change the interval between electrodes, necessity in adjusting the interval between electrodes occurs at every measurement and efficient muscle potential measurement is hindered.

Furthermore, in the muscle potential measurement device in a form where electrodes are arranged on a mount band, in general, the wearable band is stretchable and thus it is difficult to keep an appropriate interval between electrodes and there is a risk that the interval between electrodes would vary even during measurement. As described above, it is difficult to keep the interval between electrodes of the muscle electrode measurement device and, as a result, it is difficult to sufficiently increase the sensitivity in measuring a muscle potential.

### SUMMARY

According to an aspect of an embodiment, a muscle potential measurement device includes: a measurement card including a plurality of interconnects on which pads are formed; a plurality of electrodes that are connected respectively to the pads of part of the interconnects; and a processing circuit that is detachably connected to the measurement card and that is configured to measure a potential that occurs in a subject of measurement with which one ends of the electrodes make contact.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating an overview of a muscle potential measurement device according to an embodiment;

FIG. 2 is a perspective view illustrating a configuration of the muscle potential measurement device according to the embodiment;

FIG. 3 is another perspective view illustrating the configuration of the muscle potential measurement device according to the embodiment;

FIG. 4 is a perspective view illustrating a configuration of a card substrate;

FIG. 5 is a perspective view illustrating a configuration of a measurement card;

FIG. 6 is a diagram illustrating a shape of an electrode:

FIG. 7 is a diagram illustrating attachment of electrodes;

FIGS. 8A to 8C are diagrams illustrating a specific example of arrangement of the electrodes;

FIG. 9 is a diagram illustrating a modification of the card substrate; and

FIG. 10 is a diagram illustrating a specific example of arrangement of the electrodes.

### DESCRIPTION OF EMBODIMENTS

An embodiment of a muscle potential measurement device and a muscle potential measurement card will be described in detail below with reference to the accompanying drawings. The embodiment does not limit the disclosure.

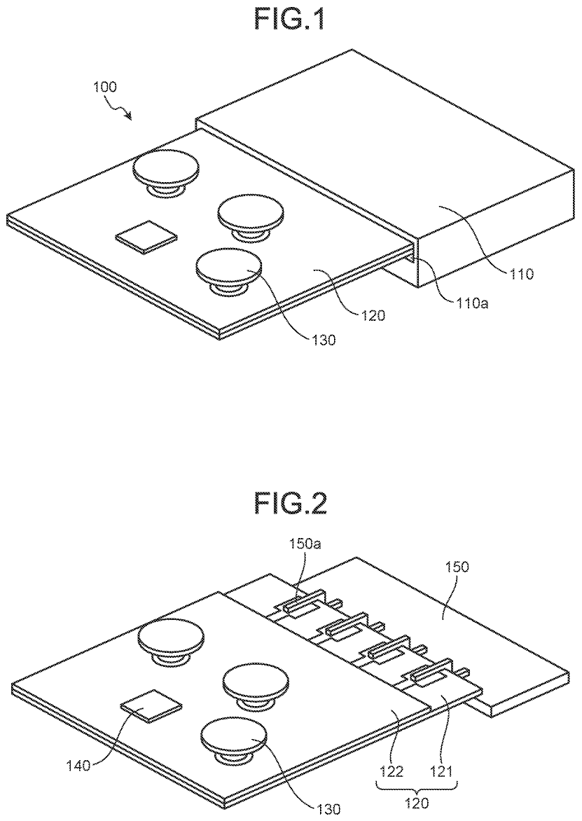

FIG. 1 is a diagram illustrating an overview of a muscle potential measurement device 100 according to an embodiment. The muscle potential measurement device 100 illustrated in FIG. 1 includes a casing 110, a measurement card 120, and electrodes 130.

The casing 110 is a case member that houses a connector that is connected to the measurement card 120 and a processing circuit. An insertion port 110a into which the measurement card 120 is insertable is formed on a surface of the casing 110 and the muscle potential measurement device 100 measures a muscle potential with the measurement card 120 being inserted into the insertion port 110a.

The measurement card 120 has one end that is insertable Into the insertion port 110a of the casing 110 and, when the one end is inserted into the insertion port 110a, the other end protrudes from the casing 110. A plurality of fixation portions to which the electrodes 130 can be fixed, respectively, are formed on the side of the other end protruding from the casing 110 and the electrodes 130 are fixed to the fixation portions that are selected to enable an appropriate interval between the electrodes. A fixation portion without an electrode fired thereto is covered with an insulating film.

The electrode 130 is a conductive member that is fired to the fixation portion of the measurement card 120 and whose end face on a side distant from the measurement card 120 is, for example, a circular plane face. The circular plane face is a contact surface that makes contact with the skin near a muscle that is a subject of measurement and, when the contact face makes contact with the skin near the muscle that is a subject of measurement, a current corresponding to a muscle potential flows into the electrode 130. The height from the surface of the measurement card 120 to the contact face is higher than the height from the surface of the measurement card 120 to an upper surface of the casing 110 and, in FIG. 1, the contact surface of the electrode 130 is at the highest position.

The electrodes 130 are fixed to the fixation portions enabling and appropriate interval between electrodes among the fixation portions that are formed on the measurement card 120. In other words, two or three electrodes 130 are fixed to the fixation portions that are selected to enable an optimum distance between electrodes according to the muscle that is a subject of measurement. The measurement card 120 with the electrodes 130 fixed thereto is inserted into the insertion port 110a of the casing 110, which makes it possible to measure a muscle potential of the muscle that is a subject of measurement with high sensitivity. To measure a potential of another muscle, replacing the measurement card 120 inserted into the insertion port 110a with the measurement card 120 on which the distance between electrodes is adjusted according to the another muscle makes it possible to measure a muscle potential of the another muscle easily.

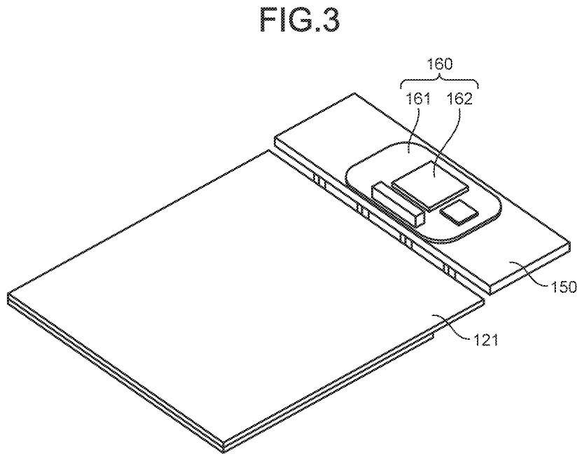

FIGS. 2 and 3 are perspective views illustrating a configuration of a muscle potential measurement device excluding the casing 110. FIG. 2 is a perspective view of the surface to which the electrodes 130 are fixed and FIG. 3 is a perspective view of a surface opposite to the surface to which the electrodes 130 are fixed.

As illustrated in FIG. 2, the measurement card 120 is formed by laminating a resist layer 122 on a card substrate 121. A plurality of interconnects are formed on the card substrate 121 and pads that are connected to a connector 150 are formed on one ends of the respective interconnects. In other words, a plurality of terminals 150a of the connector 150 are connected respectively to the pads that are formed at one end of the card substrate 121. The card substrate 121 has a rectangular shape whose one side is, for example, 20 mm to 70 mm.

The resist layer 122 is an insulating layer that is made of a resist material and that is laminated on the card substrate 121. The resist layer 122 covers the interconnects excluding pad portions that are connected to the terminals 150a. Openings for connecting the electrodes 130 to the interconnects of the card substrate 121 are formed at the center of the resist layer 122. In other words, openings penetrating through the resist layer 122 are formed in the fixation portions to which the electrodes 130 are fixed and the interconnects that are formed on the card substrate 121 are partly exposed from the openings. The electrodes 130 are connected to the interconnects on the card substrate 121 through the openings of the resist layer 122 and the currents caused by the muscle potential flow from the electrodes 130 into the connector 150 via the interconnects of the card substrate 121.

The electrodes 130 are conductive members that are fixable to the fixation portions of the measurement card 120. In other words, the electrodes 130 are adhered to the interconnects of the card substrate 121 that are exposed from the openings of the resist layer 122 with, for example, a conductive adhesive, such as a silver paste. The fixation portions to which the electrodes 130 are fixed are fixation portions that are selected to enable an appropriate distance between electrodes according to the muscle that is a subject of measurement and it is possible to change arrangement of the electrodes 130 according to each measurement card 120.

An insulating film 140 is an insulating member that is attached to the fixation portion of the measurement card 120. In other words, the insulating film 140 is attached to the fixation portion to which the electrode 130 is not fixed among the fixation portions of the measurement card 120, thereby covering the opening of the resist layer 122. Accordingly, in the fixation portion to which the electrode 130 is not fixed, the interconnect of the card substrate 121 is not partly exposed from the opening of the resist layer 122.

The connector 150 is detachably connected to the measurement card 120 in the casing 110 and transmits a current flowing from the measurement card 120 to the processing circuit. In other words, the connector 150 is provided with the terminals 150a corresponding respectively to the interconnects of the card substrate 121 and, when the measurement card 120 is inserted into the insertion port 110a of the casing 110, the terminals 150a are connected to the pads of the respective interconnects and thus the currents of the respective interconnects flow into a processing circuit 160 that is mounted on a back surface of the connector 150.

As illustrated in FIG. 3, the processing circuit 160 is mounted on the back surface of the connector 150 and the processing circuit 160 measures magnitudes of the currents flowing from the card substrate 121, thereby measuring a muscle potential. The processing circuit 160 includes a circuit board 161 and an integrated circuit (IC) chip 162. The circuit board 161 is a substrate that is mounted on the back surface of the connector 150 and various members are mounted on the substrate. The IC chip 162 is mounted on the circuit board 161 and measures magnitudes of the currents flowing from the card substrate 121, thereby measuring a muscle potential.

Note that the processing circuit 160 may include, in addition to the IC chip 162, a radio frequency (RF) IC that transmits a measured value of a muscle potential by radio and a power supply, such as a battery.

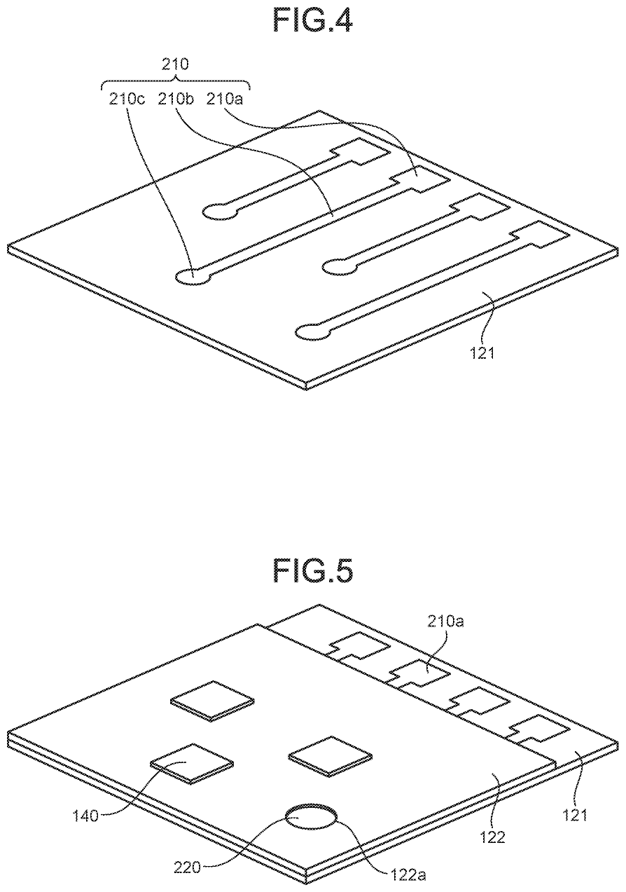

A configuration of the measurement card 120 will be described in detail next. FIG. 4 is a perspective view illustrating a configuration of the card substrate 121 that the measurement card 120 includes.

A plurality of interconnects 210 are formed on a surface of the card substrate 121. A pad 210a that is connected to the terminal 150a of the connector 150 is formed at one end of each of the interconnects 210 and a pad 210c that is connected to the electrode 130 is formed at the other end. The pad 210a and the pad 210c are connected via a linear interconnect 210b. The interconnect 210 may be formed by forming a conductive pattern of copper, a copper alloy, or the like, by, for example, photolithography.

The pads 210a that are connected to the connector 150 are formed aligned at the one end of the card substrate 121. On the other hand, the pads 210c that are connected to the electrodes 130 are not necessarily formed alignedly and, for example, the pads 210c of the interconnects 210 that are adjacent to each other may be formed in a position closer to the pad 210a and a position more distant from the pad 210a. In other words, the linear interconnects 210b of the respective interconnects 210 may have different lengths. The linear interconnect 210 of each of the interconnects 210 need not necessarily be linear and may have a bent shape or a curve shape. In association with this, it is possible co flexibly set arrangement of the pad 210c of each of the interconnects 210 and form the measurement card 120 that enables a variety of distances each between electrodes.

FIG. 5 is a perspective view illustrating a configuration of the measurement card 120 that is formed by laminating the resist layer 122 on the card substrate 121.

The resist layer 122 covers the interconnects 210 excluding the portions of the pads 210a of the card substrate 121. An opening 122a is formed in the resist layer 122 at the portion of the pad 210c of the card substrate 121. The opening 122a has a shape corresponding to a cross-sectional shape of a proximal end of the electrode 130 that is connected to the pad 210c, which alignment of the electrode 130 when the electrode 130 connected to the pad 210c.

The opening 122a allows the pad 210c of the card substrate 121 to be exposed through the resist layer 122 and a conductive adhesive 220 that is semi-cured is app led to the pad 210c. The conductive adhesive 220 is a past epoxy resin containing conductive fillers of, for example, gold, silver, or the like, and cures when heated at a curable temperature of, for example, 80 to 100 degrees. When the electrode 130 is fixed to the measurement card 120, because the measurement card 120 is heated to a temperature at which the conductive adhesive 220 cures, it is preferable that the material of the resist layer 122 and the insulating film 140 have heat-resistance durable to the temperature of curing.

The semi-cured conductive adhesive 220 is applied to the pads 210c that are exposed from the respective openings 122a, which makes it possible to easily connect the electrodes 130 to the respective pads 210c. The insulating films 140 are attached around the openings 122a into which the conductive adhesive 220 is applied. In other words, the conductive adhesive 220 that is applied to each of the pads 210c and with which the openings 122a are filled is covered with the insulating films 140. Accordingly, the conductor is not exposed to the surface of the resist layer 122 and it is possible to protect the interconnects 210 of the card substrate 121 and the conductor containing and the conductive adhesive 220 and prevent a short circuit in the case where the electrodes 130 are fixed to the measurement card 120.

When a muscle potential is measured with the measurement card 120 configured as described above, part of the pads 210c corresponding to an appropriate interval between electrodes that matches the muscle that is a subject of measurement is selected. The insulating films 140 covering the openings 122a in the positions of the selected pads 210c are peeled off and heat is applied in the state where the proximal ends of the electrodes 130 are inserted into the openings 122a so that the conductive adhesive 220 in the openings 122a cures. Accordingly, the electrodes 130 that are arranged with an appropriate interval between electrodes are fixed to the measurement card 120. The measurement card 120 to which the electrodes 130 are fixed is inserted from the side of the pads 210a into the casing 110 and accordingly the measurement card 120 is connected to the processing circuit 160 so that the muscle potential measurement device that measures a muscle potential by making the electrodes 130 make contact with the skin is obtained.

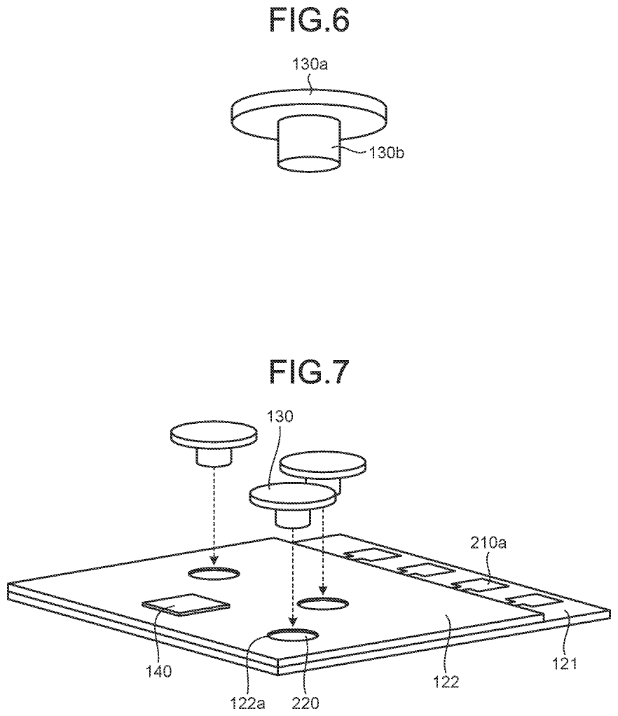

FIG. 6 is a diagram illustrating the shape of the electrode 130. The electrode 130 includes a plane portion 130a having a large diameter and having a contact surface that makes contact with the skin and a proximal end 130b having a smaller diameter than that of the plane portion 130a.

The plane portion 130a has, for example, the contact surface that is circular and whose diameter is approximately 10 mm. On the other hand, the proximal end 130b supporting the plane portion 130a has, for example, a cylindrical shape with a bottom surface whose diameter is approximately 5 mm. The electrode 130 may be formed by performing a silver/silver-chloride treatment on the surface of the resin that is formed into the shape illustrated in FIG. 6 or may be formed entirely from, for example, a conductor, such as stainless steel (SUS).

The proximal end 130b of the electrode 130 in such a shape is inserted into the opening 122a of the resist layer 122, for example, as illustrated in FIG. 7 and makes contact with the semi-cured conductive adhesive 220. In the state where the electrode 130 is pressed in a direction to the conductive adhesive 220, the conductive adhesive 220 is heated to the temperature of curing so that the electrode 130 is fixed to the measurement card 120. In other words, the electrode 130 is electrically connected to the pad 210c of the card substrate 121.

The electrode 130 need not necessarily be fixed to the pad 210c by the conductive adhesive 220. In other words, the electrode 130 may be fixed to the pad 210c, for example, by a mechanical member, such as a button or a clip. Specifically, a protruding button and a convex button may be formed on a bottom surface of the proximal end 130b of the electrode 130 and the pad 210c, respectively, and the protruding button may be fitted in the concave button and thus the electrode 130 may be fixed to the pad 210c. For example, the electrode 130 may be fixed to the pad 210c in a way that the proximal end 130b of the electrode 130 is formed into a shape of a clip capable of pinching a protrusion, a protrusion is formed on the pad 210c, and the proximal end 130b pinches the protrusion of the pad 210c.

Two or three electrodes 130 are fixed to the single measurement card 120. The electrodes 130 are connected to the pads 210c enabling an appropriate distance between electrodes among the pads 210c that are formed on the card substrate 121. Four interconnects 210 are formed on the card substrate 121 according to the embodiment and four pads 210c are formed and thus the electrodes 130 are connected to two or three of the pads 210c.

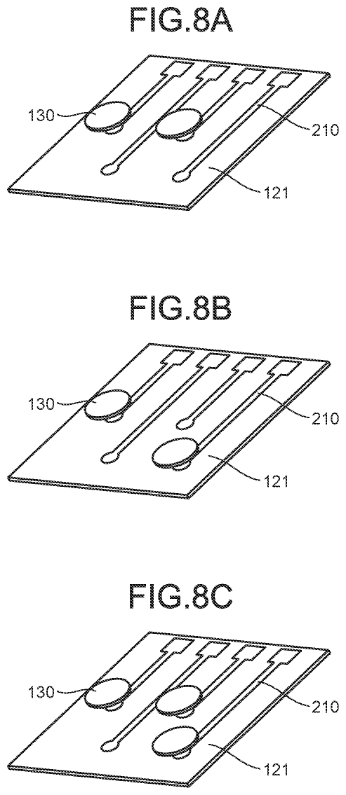

Specifically, for example, in the case where the pads 210c of short two of the interconnects 210 are in positions corresponding to an appropriate interval between electrodes according to a muscle that is a subject of measurement, as illustrated in FIG. 8A, the electrodes 130 are connected to these pads 210c. For example, when the pads 210c of the interconnects 210 on both ends are in positions corresponding to an appropriate interval between electrodes according to a muscle that is a subject of measurement, as illustrated in FIG. 8B, the electrodes 130 are connected to these pads 210c. Furthermore, in the case where three electrodes 130 containing one ground electrode are used, for example, as illustrated in FIG. 8C, the electrodes 130 are connected to the three of the pads 210c.

As described above, the interconnects 210 to which the electrodes 130 are connectable are formed on the measurement card 120 and the electrodes 130 are connectable to the pads 210c of the respective interconnects 210 with the conductive adhesive 220. Accordingly, it is possible to fix the electrodes 130 easily to the pads 210c of the interconnects 210 enabling an appropriate distance between electrodes according to a muscle that is a subject of measurement and sufficiently increase sensitivity in measuring a muscle potential. Fixing the electrodes 130 to the measurement card 120 for the first time of measurement makes it possible to measure a muscle potential efficiently without requiring adjustment on the positions of the electrodes 130 at the second and following measurement on the same muscle.

As described above, according to the present embodiment, the measurement card in which the interconnects to which the electrodes are connectable respectively are formed on the card substrate, the pads for connecting electrodes are formed on one ends of the respective interconnects, and the semi-cured conductive adhesive is applied to the pads is provided. Thus, the electrodes are connected to the pads of the measurement card to enable an optimum distance between electrodes according to a muscle that is a subject of measurement and the measurement card is connected to the processing circuit via the connector, which makes it possible to measure a muscle potential. At the measurement, because the electrodes are arranged such that an interval between electrodes according to the muscle that is a subject of measurement is enabled, it is possible to sufficiently increase sensitivity in measuring a muscle potential.

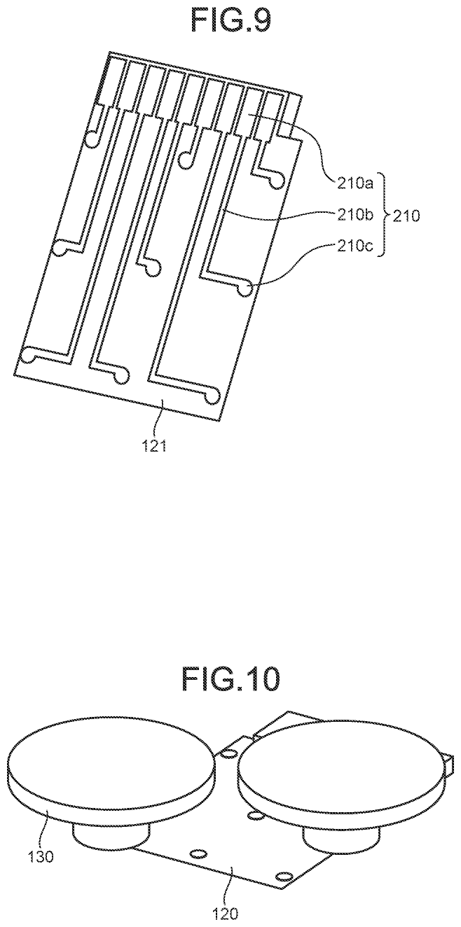

The measurement card 120 may have a size according to standards of, for example, SD memory cards. In this case, for example, as illustrated in FIG. 9, the pads 210a for connecting the connector are formed in positions according to the standards on the card substrate 121 and the pads 210c for connecting electrodes are formed on the respective pads 210c via the liner interconnects 210b. Nine interconnects 210 are formed on the card substrate 121 here.

For example, as illustrated in FIG. 10, the electrodes 130 may be fixed to a surface on the opposite side to the surface on which the interconnects 210 of the measurement card 120 are formed. In other words, the pads for connecting electrode are formed also on the back surface of the pad 210c of the card substrate 121 and the pads on both the surfaces are connected via vias, which makes it possibly to fix the electrodes 130 to the surface on the back of the surface on which the pads 210a and the liner interconnects 210b are formed. The measurement card 120 to which the electrodes 130 are fixed is connected to the connector in a shape according to standards and thus, as in the case of the above-described embodiment, it is possible to form a muscle potential measurement device that increases sensitivity in measuring a muscle potential.

In the above-described embodiment, the single measurement card 120 is inserted into the casing 110 and it is also possible to form a plurality of insertion ports 110a in the casing 110 to insert a plurality of measurement cards 120 at a time. This makes it possible to measure muscle potentials of muscles in a plurality of sites.

In the above-described embodiment, the semi-cured conductive adhesive 220 is applied to the pads 210c, and the conductive adhesive 220 may be applied to the bottom surfaces of the proximal ends 130b of the electrodes 130. Accordingly, the conductive adhesive 220 is not attached to the pad 210c to which the electrode 130 is not connected and it is possible to avoid unnecessary use of the conductive adhesive 220.

According to a mode of the muscle potential measurement device and the muscle potential measurement card that are disclosed by the present application, an effect that it is possible to sufficiently increase sensitivity in measuring a muscle potential is achieved.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A muscle potential measurement device comprising:
a measurement card including a plurality of interconnects on which pads are formed;
a plurality of electrodes that are connected respectively to the pads of part of the interconnects; and
a processing circuit that is detachably connected to the measurement card and that is configured to measure a potential that occurs in a subject of measurement with which one ends of the electrodes make contact.

2. The muscle potential measurement device according to claim 1, wherein the measurement card includes
a card substrate that has the interconnects; and
an insulating sheet that is laminated on the card substrate and that covers the interconnects.

3. The muscle potential measurement device according to claim 2, wherein the insulating sheet has openings that are formed in positions corresponding to the pads.

4. The muscle potential measurement device according to claim 3, wherein the electrodes are connected to the pads via the openings of the insulating sheet.

5. The muscle potential measurement device according to claim 3, further comprising an insulating film that covers an opening in a position corresponding to a pad other than the pads of the part of the interconnects among the openings that are formed in the insulating sheet.

6. The muscle potential measurement device according to claim 1, wherein the electrodes are connected to the pads of the part of the interconnects with a conductive adhesive that is applied to the pads.

7. The muscle potential measurement device according to claim 1, further comprising a connector that includes terminals that are detachably connected to the respective interconnects and that connects the measurement card and the processing circuit.

8. A muscle potential measurement card comprising:
a card substrate including a plurality of interconnects on which pads are formed;
an insulating sheet that is laminated on the card substrate and that convers the interconnects and in which openings into which electrodes are insertable are formed in positions corresponding to the pads; and
an insulating film that covers the opening and that is peelable.

9. The muscle potential measurement card according to claim 8, further comprising a semi-cured conductive adhesive that is applied to the pad in the opening of the insulating sheet,
wherein the insulating film covers the conductive adhesive.
